(19) United States
(12) Patent Application Publication
(10) Pub. No.: US 2026/0183225 A1
(43) Pub. Date: Jul. 2, 2026

(54) PHARMACEUTICAL COMPOSITION FOR TOPICAL ADMINISTRATION COMPRISING A VOLATILE ANAESTHETIC

(71) Applicant: LABORATORIOS GEBRO PHARMA, S.A., Barcelona (ES)

(72) Inventors: Franz HÄUSLER, Bad Reichenhall (DE); Marta MUÑOZ TUDURÍ, Barcelona (ES); Marc CENTELLAS CASADO, Barcelona (ES)

(21) Appl. No.: 18/865,585

(22) PCT Filed: May 13, 2022
(86) PCT No.: PCT/EP2022/063007

§ 371 Date: Nov. 13, 2024

(51) Int. Cl. A61K 9/00; A61K 9/06; A61K 31/08; A61K 47/02; A61K 47/10; A61K 47/14; A61K 47/44; A61P 23/02

(52) U.S. Cl. CPC A61K 9/0014; A61K 9/06; A61K 31/08; A61K 47/02; A61K 47/10; A61K 47/14; A61K 47/44; A61P 23/02

(57) ABSTRACT

A pharmaceutical composition for topical administration comprises at least one volatile anaesthetic, at least one pharmaceutically acceptable lipophilic solvent and a lipophilic formulation base.

### CROSS REFERENCE

This application is a U.S. National Phase Application of International Application No. PCT/EP2022/063007 filed May 13, 2022, the entire disclosure of which is hereby incorporated by reference herein.

### TECHNICAL FIELD

The present disclosure relates to the field of pharmaceutical compositions comprising volatile anaesthetics and to their use. In particular, the present disclosure provides a pharmaceutical composition comprising a volatile anaesthetic, wherein the pharmaceutical composition is formulated for topical administration.

### BACKGROUND ART

Both acute and chronic pain are a widespread problem with a wide variety of causes.

Pain can be classified as either nociceptive pain or neuropathic pain, according to clinical characteristics and assumed underlying mechanisms. However, in practice this distinction is not always applicable as patients may feature mixed pain or mixed pain perceptions, including both nociceptive and neuropathic pain characteristics.

Nociceptive pain is a normal response of the body to potential harm and serves to protect from dangers that are encountered. It can be acute or chronic in nature, depending on the specific case and the cause of the pain. Nociceptive pain can be further classified into subtypes as somatic or visceral pain depending on the origin. Somatic nociceptive pain originates from pain receptors located on the surface of the body or in the musculoskeletal tissues. This type of pain is usually localized to a certain area of the body and often worsens with activity; however, it is typically relieved by periods of rest. Visceral pain refers to pain that results from pain receptors located in the internal organs of the body. This type of pain is usually described as a deep, pressure-like feeling that is not localized to a certain point in the body.

Neuropathic pain is caused by injuries of the nerves that are involved in the transmission of electrical signals sending messages of pain from the receptors to the brain. This type of pain is typically described as a sharp, shooting pain and is often more intense as compared to other types of pain. There are various causes of nerve damage and resulting neuropathic pain. Some conditions that may have peripheral and localized neuropathic pain among their symptoms are painful diabetic neuropathy, posttherapeutic neuralgia,

trigeminal neuralgia, lumbar radiculopathy, polyneuropathy caused by the immunodeficiency virus human (HIV), alcoholic neuropathy, post-surgical neuropathy, B12 deficiency neuropathy or cancer neuropathy (induced by chemotherapy, secondary to cancer antigens, or induced by tumour infiltration or compression of structures). In association with neuropathic pain, allodynia may occur. Allodynia is a condition in which pain is caused by a stimulus that does not normally elicit pain. In patients suffering from allodynia quality of life is further reduced, as even minor stimuli lead to a strong sensation of pain. In general terms, the treatment strategy and the drugs used in different types of pain are not the same.

For nociceptive pain, the first-line management is a treatment with analgesic medications, such as NSAIDs. For more severe pain, opioids can often help to provide effective pain relief.

Neuropathic pain is frequently resistant to treatment and if an effect is observed it may be transient. NSAIDs are generally ineffective for the treatment of neuropathic pain. Several classes of active ingredients have approved indications for neuropathic pain, including anticonvulsants (gabapentinoids, carbamazepine), antidepressants (tricyclic antidepressants, SNRIs), or topically applied lidocaine or capsaicin. However, they have variable efficacy and are often associated with severe side effects.

Those who suffer from pain have a severely reduced quality of life. Pain often is associated with feelings of depression, lack of concentration, lower energy levels and loss of enjoyment of daily life. Pain can be difficult to treat as it is a complex problem with physical, emotional and social components. Currently available pain medication cannot always sufficiently control the pain or is associated with adverse side effects. Another problem with pain management is opioid dependence. Accordingly, there is a need for improved treatment options.

Volatile anaesthetics have already been investigated for local pain reduction. Volatile anaesthetics are inhalational anaesthetics that are liquid at room temperature, but easily evaporate due to their high vapour pressure. They all share the property of being lipophilic and poorly soluble in water.

US2019358173 discloses local or regional delivery of a volatile anaesthetic, particularly as intrathecal or epidural delivery, wherein the volatile anesthetic is provided in an aqueous based solution to a patient in an amount effective to reduce chronic or acute pain. These routes of administration require medical training and are not easily available in everyday settings.

WO 2014/143964 suggests volatile anaesthetics for local use and provides compositions for topical use, particularly as an ointment, solution or gel. However, the volatile anaesthetics easily evaporate from these compositions and are lost to the environment. Since the volatile anaesthetic can only work as long as it is in direct contact to the skin a fast evaporation reduces duration of action. To reach for a sufficient period of time an effective amount of the volatile anaesthetic at the site of action the initial concentration in these composition needs to be very high, typically >50 wt %, and the frequency of application has to be high. This results in high costs for the active ingredients. More importantly, inhalation of the evaporating amount of the volatile anaesthetic may be harmful to patients, and high concentrations have been shown to cause irritation to the skin. Furthermore, volatile anaesthetics have a high greenhouse potential and their emission should be minimized. In addition, these compositions are not very stable during storage and have a short shelf life. However, to improve quality of life for patients suffering from pain, it is not only necessary to sufficiently control the pain and avoid side effects but also to provide an efficient well tolerated treatment, in particular a treatment being effective and having a long-lasting effect. In addition, it is also important that the therapy is cost-efficient and environmentally sustainable.

### SUMMARY OF INVENTION

It is the objective of the present disclosure to provide a composition for topical administration, in particular for use in a method to reduce pain, wherein the composition is effective, long-lasting, well-tolerated and sustainable.

The objective is solved by the subject of the claims and as further described herein.

In one aspect, the present disclosure provides a pharmaceutical composition for, e.g. formulated for, topical administration, comprising at least one volatile anaesthetic, at least one pharmaceutically acceptable lipophilic solvent and a lipophilic formulation base. In certain embodiments the volatile anaesthetic is selected from the group consisting of isoflurane, halothane, enflurane, sevoflurane, desflurane, methoxyflurane, and mixtures thereof.

According to certain embodiments the pharmaceutical composition comprises up to 40 wt % of the volatile anaesthetic, in particular 5 wt % to 35 wt % of the volatile anaesthetic, more particularly 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23, wt %, 24 wt %, or 25 wt % of the volatile anaesthetic. In certain embodiments the volatile anaesthetic is sevoflurane, and wherein it is particularly provided that the pharmaceutical composition comprises up to 40 wt % sevoflurane, in particular 5 wt % to 35 wt % sevoflurane, more particularly 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23, wt %, 24 wt %, or 25 wt % sevoflurane.

In certain embodiments the pharmaceutical composition is a semi-solid formulation, particularly an ointment, wherein the lipophilic formulation base in particular is an ointment base. In certain embodiments the pharmaceutical composition is a single-phase formulation, more particularly a lipophilic single-phase formulation. In particular in the single-phase formulation the volatile anaesthetic is completely dissolved in the lipophilic formulation base and the lipophilic solvent acts as a solubilizer, enabling the solution of the volatile anaesthetic in the lipophilic formulation base.

In certain embodiments the lipophilic solvent is an ester of a fatty acid, more particularly an ester of a fatty acid selected from the group consisting of myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, caprylic acid, capric acid, lauric acid, myristic acid, palmitic acid, stearic acid, arachidic acid, behenic acid, lignoceric acid, cerotic acid, and any combinations thereof.

According to certain embodiments the lipophilic solvent is an ester of a fatty acid as described before and a low-molecular alcohol, in particular a C₁, C₂, C₃, C₄, C₅, or C₆ alcohol, more particularly n-propanol or isopropanol.

According to certain embodiments the pharmaceutical composition comprises up to 45 wt % of the lipophilic solvent, in particular 10 wt %, 11 wt %, 12 wt %, 13 wt %, 14 wt %, 15 wt %, 16 wt %, 17 wt %, 18 wt %, 19 wt % or 20 wt % of the lipophilic solvent.

In certain embodiments the lipophilic solvent is isopropyl myristate, wherein it is particularly provided that the pharmaceutical composition comprises up to 45 wt % isopropyl myristate, in particular 10 wt %, 11 wt %, 12 wt %, 13 wt %, 14 wt %, 15 wt %, 16 wt %, 17 wt %, 18 wt %, 19 wt % or 20 wt % isopropyl myristate.

In certain embodiments the lipophilic formulation base is a natural or artificial pharmaceutically acceptable semi-solid hydrocarbon compound or mixture of hydrocarbon compounds with a drop point between 35° C. und 70° C., particularly petroleum jelly and/or a mixture of polyethylene and mineral oil, more particularly hydrocarbon gel 50 W or hydrocarbon gel 55 W.

In certain embodiments the pharmaceutical composition comprises 35 wt % to 90 wt % of a pharmaceutically acceptable semi-solid hydrocarbon compound or mixtures of hydrocarbon compounds, particularly 50 wt %, 51 wt %, 52 wt %, 53 wt %, 54 wt %, 55 wt %, 56 wt %, 57 wt %, 58 wt %, 59 wt %, 60 wt %, 61 wt %, 62 wt %, 63 wt %, 64 wt %, 65 wt %, 66 wt %, 67 wt %, 68 wt %, 69 wt %, 70 wt %, 71 wt %, 72 wt %, 73 wt %, 74 wt % or 75 wt % 5 petroleum jelly.

According to certain embodiments the lipophilic solvent works as a solubilizer, enabling the solution of the volatile anaesthetic in the lipophilic formulation base.

In certain embodiments the pharmaceutical composition comprises at least one pharmaceutically acceptable stabilizer, particularly a fatty alcohol.

In certain embodiments the pharmaceutical composition comprises 0.1 wt % to 5 wt % of the stabilizer, particularly 3 wt % to 4 wt % of the stabilizer. 5 F″

According to certain embodiments the pharmaceutical composition comprises a fatty alcohol that is a C₁₀ to C₃₀ alcohol, particularly cetyl alcohol, stearyl alcohol or a mixture thereof, wherein it is particularly provided that the pharmaceutical composition comprises 1 wt % to 5 wt %, more particularly 3 wt % to 4 wt %, of the fatty alcohol.

In certain embodiments the pharmaceutical composition comprises at least one carrier, particularly a solid carrier, more particularly selected from the group consisting kaolin, bentonite, hectorite, colloidal magnesium, aluminum silicate, silicon dioxide, magnesium trisilicate, aluminum hydroxide, magnesium hydroxide, magnesium oxide or talc.

In certain embodiments the pharmaceutical composition comprises 1 wt % to 15 wt % of the solid carrier, particularly 3 wt % to 7 wt % of the solid carrier, more particularly 5 wt % of the solid carrier, wherein it is particularly provided that the solid carrier is talc.

According to certain embodiments the pharmaceutical composition comprises or consists exclusively of
Sevoflurane 10.0-25.0 wt %
Isopropyl Myristate 10.0-20.0 wt %
Talc 3.0-7.0 wt %
Petroleum jelly 55.0-75.0 wt %
Cetyl alcohol 2.0-5.0 wt %

In a further aspect, the present disclosure provides the pharmaceutical composition for use in a method for treatment.

In certain embodiments the pharmaceutical composition is provided for use in a method for treatment of pain, wherein the pharmaceutical composition is administered topically, in particular cutaneously.

According to certain embodiments, after administration, the site of administration is covered by an occlusion-producing layer, particularly by an adhesive bandage or bandage.

In certain embodiments the pharmaceutical composition is provided for use in a method for the treatment of peripheral neuropathic pain. In certain embodiments the pharmaceutical composition is provided for use in a method for the treatment of an ulcer.

According to certain embodiments the pharmaceutical composition is applied onto an open wound.

In certain embodiments the pharmaceutical composition is applied to the site of a body of a subject in need where the pain is perceived.

In certain embodiments the pharmaceutical composition is applied in an amount effective to reduce pain. In certain embodiments the pharmaceutical composition is applied once or twice a day, more particularly every 12 to 24 hours.

In an even further aspect, the present disclosure provides sevoflurane for use in a method for the treatment of pain. In certain embodiments sevoflurane for use in a method for the treatment of pain is administered topically, particularly cutaneously. More particularly, sevoflurane for use in a method for the treatment of pain is administered in form of a pharmaceutical composition as described before.

According to certain embodiments, after administration of sevoflurane, the site of administration is covered by an occlusion-producing layer, particularly by an adhesive bandage or bandage.

In certain embodiments sevoflurane is provided for use in a method for the treatment of peripheral neuropathic pain.

In certain embodiments sevoflurane is provided for use in a method for the treatment of an ulcer.

In certain embodiments sevoflurane is applied onto an open wound.

In certain embodiments sevoflurane is applied to the site of a body of a subject where the pain is perceived. In certain embodiments the subject is a mammal, in particular a human, mouse or rat.

According to a certain embodiment, sevoflurane wherein is applied in an amount effective to reduce pain.

In certain embodiments sevoflurane is administered by injection, more particularly by subcutaneous injection.

In certain embodiments sevoflurane is applied once or twice a day, more particularly every 12 to 24 hours.

### BRIEF DESCRIPTION OF DRAWINGS

For the purpose of illustration, certain embodiments of the invention are illustrated in the drawings. However, the invention is not limited to the specific formulations of said examples.

FIG. 1 is a series of graphs showing the evaporation rates of a certain embodiment of the pharmaceutical composition compared to prior art formulations during 1 hour.

FIG. 2 is a series of graphs showing the evaporation rates of a certain embodiment of the pharmaceutical composition compared to prior art formulations during 24 hours.

FIG. 3 is a series of graphs showing the evaporation rates from a 500 μm layer of a certain embodiment of the pharmaceutical composition compared to prior art formulations during 1 hour.

FIG. 4 is a series of graphs showing the effect of sevoflurane in an isolated nerve fibre model.

FIG. 5 is a series of graphs showing the effect of sevoflurane in a mouse vas deferens preparation.

FIG. 6 is a series of graphs showing the effect of sevoflurane in a guinea-pig ileum LMMP.

FIG. 7 is a comparison of the results of the mouse vas deferens model tested and the guinea pig ileum LMMP model.

FIG. 8 is a series of graphs showing the anti-allodynic effect of sevoflurane in different concentrations compared to saline solution in mice.

FIG. 9 is a series of graphs showing the anti-hyperalgesic effect of sevoflurane in different concentrations compared to saline solution in mice.

FIG. 10 is a series of graphs showing the effect of sevoflurane on spontaneous pain compared to morphine.

### DETAILED DESCRIPTION

The present disclosure relates to pharmaceutical compositions formulated for topical administration, comprising at least one volatile anaesthetic, at least one pharmaceutically acceptable lipophilic solvent and a lipophilic formulation base. The pharmaceutical compositions of the present disclosure have a very low evaporation loss of the volatile anaesthetic, are stable and sustainable.

In a further aspect, the present disclosure relates to sevoflurane for use in a method for the treatment of pain. In particular the present disclosure relates to sevoflurane for use in a method for the treatment of pain, wherein sevoflurane is administered topically, particularly cutaneously. In animal models no relevant side effects of sevoflurane occurred and it was shown to be effective in nociceptive pain as well as in neuropathic pain.

The present disclosure provides a new treatment option for patients suffering from pain that is efficient without relevant adverse side effects, long lasting, cost-efficient and sustainable.

Unless defined otherwise, all technical and scientific terms used herein generally have the same meaning as commonly understood by one of ordinary skill in the art to which this present disclosure belongs.

As used herein, the term “pharmaceutical composition” refers to a mixture of at least one medicinal substance of the present disclosure with other chemical compounds, such as carriers, stabilizers, diluents, solubilizers, dispersing agents, suspending agents, thickening agents, and/or excipients. The pharmaceutical composition facilitates administration of the medicinal substance to an organism.

As used herein, the term “topical” as applied to mode of administration refers to an application at the site of action; it includes, but is not limited to “cutaneous”. The term “cutaneous” refers to the application of a composition to the skin of a subject, irrespective of whether it is intact, damaged or injured.

The term “lipophilic” represents the affinity of a substance or a composition to a lipophilic environment, e.g. a fat or hydrocarbon compounds. The lipophilicity can be measured by the distribution behaviour of a compound in a biphasic system, especially by its octanol-water partition coefficient K_{ow}. The common logarithm Log K_{ow}is positive for lipophilic and negative for hydrophilic substances.

A “solvent” is a substance that dissolves a solute, resulting in a solution. In a solution, all of the solutes are uniformly distributed at a molecular level and no residue remains. A solvent-solute mixture is a single-phase system.

As used herein, the term “single-phase formulation” or “single-phase system” is used to distinguish the formulation or system from an emulsion formulation or system, consisting of a lipophilic and a hydrophilic phase either in form of an O/W (oil in water) or a W/O (water in oil) system. The term does not exclude that a part of the molecules is present in a solid state and another part in a liquid state, as this is a typical feature of semi-solid compounds such as petroleum jelly.

“Semi-solid” formulations are formulations with a semi-solid consistency and are defined in the Ph. Eur.

The “dropping point” is the temperature at which a compound or formulation passes from a semi-solid to a liquid state under specific test conditions according to the Ph. Eur.

As used herein, a “formulation base” is a vehicle into which active ingredients and functional excipients may be incorporated. The formulation base contributes significantly to the consistency of the composition and often accounts for the largest proportion by weight of a composition.

As used herein, the term “treatment” refers to a care given to a patient to reduce the negative impact of an illness or injury on the patient. “Effective” means adequate to accomplish a desired, expected, or intended result.

As used herein, the term “subject” or “individual” or “patient” shall refer to a warm-blooded mammalian, particularly a human being e.g., an adult or child (including newborns). The subject may be a patient in need of treatment of a disease described herein.

As used herein, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unmentioned elements or method steps. In contrast thereto, “consisting” is considered as a closed definition without further elements. Thus, “comprising” is broader and contains the “consisting” definition.

Every quantity defined herein is meant to refer to the actual given value, and it is also meant to refer to the approximation to such given value that would reasonably be inferred based on the ordinary skill in the art, including approximations due to the experimental and/or measurement conditions for such given value. Throughout this disclosure, various aspects of this disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible sub-ranges as well as individual numerical values within that range.

The term “pharmaceutically acceptable” as used herein refers to a material, such as a carrier or diluent, which does not abrogate the biological activity or properties of the active ingredient, and is relatively non-toxic, i.e., the material may be administered to an individual without causing undesirable biological effects or interacting in a deleterious manner with any of the components of the composition in which it is contained.

The pharmaceutical composition in specific embodiments particularly comprises a volatile anaesthetic that is selected from the group consisting of isoflurane, halothane, enflurane, sevoflurane, desflurane, methoxyflurane, and mixtures thereof. These compounds have been shown to be save and have few side effects.

According to specific embodiments the pharmaceutical composition comprises up to 40 wt % of the volatile anaesthetic, in particular 5 wt % to 35 wt % of the volatile anaesthetic, more particularly 10 wt % to 25 wt % of the volatile anaesthetic. Such a pharmaceutical composition provides an effective and cost-efficient treatment option and is sufficiently stable to reduce the environmental impact of the treatment. The stability is also sufficient for storage of the composition.

In particularly advantageous embodiments the volatile anaesthetic is sevoflurane. Sevoflurane is a highly fluorinated methyl isopropyl ether with the chemical formula C₄H₃F₇O and CAS Number 8523-86-6. It is used as an inhalation anaesthetic agent, having a light pleasant odour, while the smell of other volatile anaesthetics can be perceived as pungent. It has a GWP₂₀ value of 440. Herein, Sevoflurane was tested under the internal code LGP10-16.

The greenhouse potential of compounds is expressed by the Global Warming Potential (GWP), e.g. relating to 20 years (GWP₂₀). The GWP₂₀of sevoflurane is 440, meaning that sevoflurane is 440 times more potent than CO₂. The respective values for Isoflurane are 1800 and for Desflurane 6810. Sevoflurane has therefore the lowest greenhouse effect of this class of compounds.

It is particularly provided that the pharmaceutical composition comprises up to 40 wt % sevoflurane, in particular 5 wt % to 35 wt % sevoflurane, more particularly 10 wt %, to 25 wt % sevoflurane. Said specific pharmaceutical compositions are effective with a fast onset and a sufficient duration of action and are stable.

To advantageously improve the handling of the pharmaceutical composition in specific embodiments, the composition is a semi-solid formulation. This simplifies the administration. For the present disclosure it is particularly suitable if the composition is an ointment, as a lipophilic ointment can reduce evaporation of the volatile anaesthetic and improve stability of the composition. It is therefore particularly suitable if the lipophilic formulation base is an ointment base.

In specific embodiments the pharmaceutical composition is a single-phase formulation, more particularly a lipophilic single-phase formulation. This further increases the stability of the composition. The single-phase formulation is characterized by the fact that the volatile anaesthetic is completely dissolved in the lipophilic formulation base and the lipophilic solvent acts as a solubilizer, enabling the solution of the volatile anaesthetic in the lipophilic formulation base.

In specific embodiments the lipophilic formulation base is a natural or artificial pharmaceutically acceptable semi-solid hydrocarbon compound or mixture of hydrocarbon compounds with a dropping point between 35° C. and 70° C. This further reduces evaporation. In some embodiments the lipophilic formulation base is petroleum jelly or a mixture of polyethylene and mineral oil, more particularly hydrocarbon gel 50 W or hydrocarbon gel 55 W. Hydrocarbon gel 50 W or hydrocarbon gel 55 W are available as Plastibase®.

The administration is improved for specific embodiments of the pharmaceutical composition that comprise 35 wt % to 90 wt %, in particular 50 wt % to 75 wt %, of the pharmaceutically acceptable semi-solid hydrocarbon compound or mixtures of hydrocarbon compounds. Particularly suitable are compositions comprising 50 wt % to 75 wt % petroleum jelly. Such compositions are easy to dose, while at the same time evaporation of the volatile anaesthetic is reduced.

To further improve the stability of the composition, in specific embodiments, the lipophilic solvent is an ester of a fatty acid. More particularly, the solvent is an ester of a fatty acid selected from the group consisting of myristoleic acid, palmitoleic acid, sapienic acid, oleic acid, elaidic acid, vaccenic acid, linoleic acid, linolelaidic acid, α-linolenic acid, arachidonic acid, eicosapentaenoic acid, erucic acid, docosahexaenoic acid, caprylic acid, capric acid, lauric acid, myristic acid, palmitic acid, stearic acid, arachidic acid, behenic acid, lignoceric acid, cerotic acid, and any combinations thereof. The stability can be further improved, where the lipophilic solvent is an ester of a fatty acid and a low-molecular alcohol, in particular a C₁ to C₆alcohol, more particularly an ester of n-propanol or isopropanol.

To reduce the evaporation of the volatile anaesthetic, according to specific embodiments, the pharmaceutical composition comprises up to 45 wt % of the lipophilic solvent, in particular 10 wt % to 20 wt % of the lipophilic solvent.

The composition is particularly stable if the lipophilic solvent is isopropyl myristate. Best results for stability can be achieved for specific embodiments of the pharmaceutical composition that comprise up to 45 wt % isopropyl myristate, in particular 10 wt % to 20 wt % isopropyl myristate. This amount of isopropyl myristate is suitable to improve the formation of a stable single-phase system.

To further increase stability of the pharmaceutical composition, in specific embodiments, it is particularly provided that the lipophilic solvent works as a solubilizer, enabling the solution of the volatile anaesthetic in the lipophilic formulation base. A solubilizer increases the solubility of the volatile anaesthetic in the lipophilic formulation base. As a result, the composition is more stable and the evaporation of the volatile anaesthetic is reduced.

According to specific embodiments the pharmaceutical composition comprises at least one pharmaceutically acceptable stabilizer. A particularly suitable stabilizer for the present composition is a fatty alcohol.

In specific embodiments, the pharmaceutical composition particularly comprises 0, 1 wt % to 5 wt % of the stabilizer, particularly 3 wt % to 4 wt % of the stabilizer.

Specific embodiments of the pharmaceutical composition have an improved stability, when they comprise a fatty alcohol that is a C₁₀ to C₃₀ alcohol, particularly cetyl alcohol, stearyl alcohol or a mixture thereof, wherein it is particularly provided that the pharmaceutical composition comprises 1 wt % to 5 wt %, more particularly 3 wt % to 4 wt %, of the fatty alcohol.

To further increase the stability of the formulation in specific embodiments, the pharmaceutical composition particularly comprises at least one carrier, particularly a solid carrier. Particularly suitable is a solid carrier selected from the group consisting of kaolin, bentonite, hectorite, colloidal magnesium, aluminum silicate, silicon dioxide, magnesium trisilicate, aluminum hydroxide, magnesium hydroxide, magnesium oxide or talc.

The skin feel of the preparation is particularly improved in specific embodiments, wherein the pharmaceutical composition comprises 1 wt % to 15 wt % of the solid carrier, particularly 3 wt % to 7 wt % of the solid carrier, more particularly 5 wt % of the solid carrier, wherein it is particularly provided that the solid carrier is talc.

Particularly stable and effective is a pharmaceutical composition comprising or consisting of

| | | |
|---|---|---|
| Sevoflurane | 10.0-25.0 | wt % |
| Isopropyl Myristate | 10.0-20.0 | wt % |
| Talc | 3.0-7.0 | wt % |
| Petroleum jelly | 55.0-75.0 | wt % |
| Cetyl alcohol | 2.0-5.0 | wt % |

A particularly advantageous composition comprises or consists of

| | | |
|---|---|---|
| Sevoflurane | 10.0-20.0 | wt % |
| Isopropyl Myristate | 15.0 | wt % |
| Talc | 5.0 | wt % |
| Petroleum jelly | 57.0-66.5 | wt % |
| Cetyl alcohol | 3.0-3.5 | wt % |

A pharmaceutical composition as described before can be provided by a method comprising the following steps:
providing a formulation base, particularly in combination with a stabilizer, in particular providing petroleum jelly and cetyl alcohol, by melting at 60° C. and cooling down to room temperature (<25° C.) whilst stirring,
mixing the volatile anaesthetic with the lipophilic solvent,
optionally adding a solid carrier to the mixture,
adding the mixture to the formulation base,
stirring the total mixture until it is homogeneous,
optionally filling the preparation into gas tight containers, particularly into aluminium tubes.

The pharmaceutical formulation as described before can also be provided by a method comprising the following steps:
providing a lipophilic formulation base, particularly petrolatum jelly or wax, a lipophilic solvent, particularly isopropyl myristate, and a stabilizer, particularly cetyl alcohol; then melting the compounds at 60° C. and cooling down to room temperature (<25° C.) whilst stirring,
optionally adding a solid carrier, particularly talc, whilst stirring,
adding the volatile anaesthetic, particularly sevoflurane,
stirring the mixture until it is homogeneous,
optionally filling the preparation into gas tight containers, particularly into aluminium tubes.

These methods provide a homogenous and stable composition.

To avoid evaporation, the formulation should be filled into the gas tight containers immediately after preparation of the formulation.

In another aspect, the present disclosure provides the pharmaceutical composition as described above for use in a method for treatment. The composition is effective, well-tolerated and provides a cost-effective and sustainable treatment option.

The pharmaceutical composition is particularly effective for use in a method for treatment of pain, wherein the pharmaceutical composition is administered topically, in particular cutaneously.

To further increase sustainability of the treatment in certain embodiments, the site of administration is covered by an occlusion-producing layer after administration, particularly by an adhesive bandage or bandage. Thus, evaporation of the volatile anaesthetic from the composition can be further reduced. The amount of the volatile anaesthetic in the composition that is needed to achieve a therapeutical effect is reduced as well.

The pharmaceutical composition is particularly effective in a method for the treatment of peripheral neuropathic pain.

The pharmaceutical composition is also particularly effective in a method for the treatment of an ulcer.

The pharmaceutical composition is also well-tolerated, if it is applied onto an open wound.

An effective treatment is provided if the pharmaceutical composition is applied to the site of a body of a subject in need where the pain is perceived.

For particularly effective treatment, the pharmaceutical composition is applied in an amount effective to reduce pain.

An effective, well-tolerated and sustainable treatment is provided if the pharmaceutical composition is applied once or twice a day, more particularly every 12 to 24 hours.

In another aspect of the present disclosure, sevoflurane is provided for use in a method for the treatment of pain. In particular, sevoflurane is provided for use in a method, wherein sevoflurane is administered topically, particularly cutaneously. In particular, sevoflurane can be administered to the intact or damaged skin. In particular, sevoflurane is provided in form of a pharmaceutical composition as described above. The pharmaceutical composition as described above is particularly useful for cutaneous application. In this embodiment sevoflurane provides an effective treatment for a patient suffering from pain. Furthermore, sevoflurane is provided in a cost-efficient and sustainable way.

Sevoflurane for use in a method for treatment of pain can also be administered by parenteral injection, preferably by subcutaneous injection.

Specifically, sevoflurane is provided for use in a method, wherein after administration the site of administration is covered with an occlusion-producing layer, particularly with an adhesive bandage or bandage. This can further improve effectiveness of sevoflurane since evaporation is reduced.

Specifically, sevoflurane is provided for use, wherein sevoflurane is applied to the site of a body of a subject where the pain is perceived.

According to an even further specific embodiment, sevoflurane is provided for use in a method for the treatment of peripheral neuropathic pain. As neuropathic pain is difficult to treat, and no options for topical treatment are available, this provides an important new treatment option, which is cost-efficient and sustainable. Additionally, the treatment is effective, easy to administer and to dose. As a result, the quality of life of a patient can be improved significantly.

According to a specific embodiment, sevoflurane is provided for use in a method for the treatment of an ulcer. The administration of sevoflurane, particularly in a concentration of 10-25 wt %, does not interfere with healing and pain associated with the ulcer can effectively be managed to improve quality of life of a patient.

Specifically, sevoflurane is provided for use, wherein sevoflurane is applied onto an open wound. Sevoflurane, particularly in a concentration of 10-25 wt %, has been shown to be safe and effective also on open wounds. The pharmaceutical composition as described above provides for a long-lasting effect, so that applications can be made sparingly and wound healing rarely is disturbed as a result.

In yet another specific embodiment, sevoflurane is particularly provided for use, wherein sevoflurane is applied once or twice a day, more particularly every 12 to 24 hours. With this dosage regime, a lasting and effective pain relief can be achieved and the application is only associated with little effort for the patient.

### Examples

The present disclosure is further described in detail by reference to the following experimental examples. These examples are provided for purposes of illustration only and are not intended to be limiting unless otherwise specified. Thus, the present disclosure should in no way be construed as being limited to the following examples, but rather should be construed to encompass any and all variations which become evident as a result of the teaching provided herein.

### Example Set 1-In Vitro

The objective of this initial study was to establish if a pain reduction could be achieved and, if so, if the reduction of pain was due to the damage of the neurons (no pain signalling due to the toxicity of the product) or to an analgesic effect.

Further, the potential analgesic effect, dose-response effect, concentration, reversibility and duration were assessed. LGP10-16 (sevoflurane) was administered in different concentrations as dilutions.

In a first step, an isolated nerve fibre model was used and the effect of a 25% dilution (nutrient solution) of LGP10-16 on the different electrophysiological variables of the A delta nerve fibres was evaluated. The results are summarized in FIG. 4. The stimulated fibres from the A delta type correspond to nociceptive sensory fibres. 250 μl of LGP10-16 and 750 μl of nutrient solution were used in this test. The effect caused by this LGP10-16 25% dilution was an inhibition of the nerve fibre firing capacity in view of an increasing pressure stimulus. This effect lasted up to 90 minutes after a single application and was maintained after washing the fibre between one experiment and the next, which demonstrates the intensity of the effect. The sensitivity of the fibres indicated that with a 25% solution a maximum effect was reached.

The experiment suggests that LGP10-16 is capable of blocking transmission of the nociceptive signal and thus has an analgesic effect. This effect was reversible, thus no damage occurred.

In a second step, the effect of different concentrations of LGP10-16 on preparations of mouse vas deferens and guinea-pig ileum LMMP was studied.

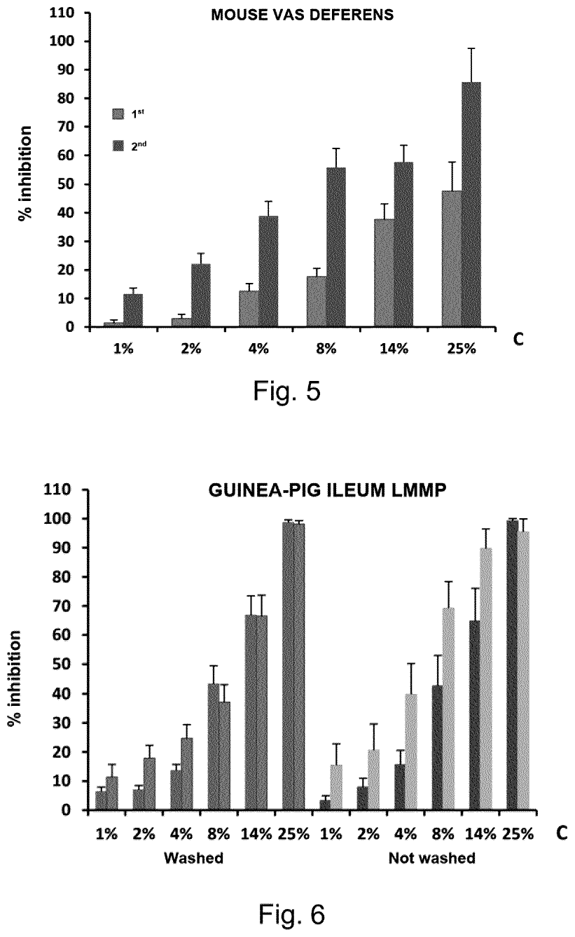

FIG. 5 shows the results of the mouse vas deferens model. The graph shows the inhibition of contractile activity of the mouse vas deferens preparation after non-accumulative addition of increasing concentrations (from 1% to 25%) of LGP10-16. After adding a concentration of the drug to the organ bath, it is left for 9 minutes to test its effect, after which the preparation is washed. At the end of the first concentration-response curve (left bars), it is left for 90 mins and the second curve is created (right bars).

In the mouse vas deferens, the administration of LGP10-16 to the bath caused a concentration-dependent inhibition of the contractile response. The effect of LGP10-16 was maintained even 90 minutes after washing the preparation (i.e. after changing the nutrient solution in which the vas deferens is incubated). The response was even more significant when it was reapplied.

This can be explained by LGP10-16 having a long-lasting effect (which is also in line with the results of the isolated nerve fibre model). The effect was maintained after washing, and the second administration caused a cumulative effect in the preparation's response.

FIG. 6 shows that LGP10-16 was also effective in the guinea pig ileum LLMP model. The inhibitory effect of contractile activity by administration of increasing concentrations (1% to 25%) of LGP10-16 in this preparation was greater, reaching 100% inhibition with the first curve and maintaining similar effects after washing (90 minutes). In the case of this preparation, after washing, the curve has the same appearance, and the LGP10-16 activity is the same in both curves. To further investigate these differences, the battery of guinea-pig ileum experiments was repeated, this time without washing between the two curves, to see if the effect changed in any way. As was expected, when the preparation was not washed between the two curves, the effect of LGP10-16 (in concentrations from 1% to 25%) was somewhat greater, with a more inhibitory effect noted at intermediate doses. This is a clear synergic effect, as a result of the continued presence of LGP10-16 in the bath.

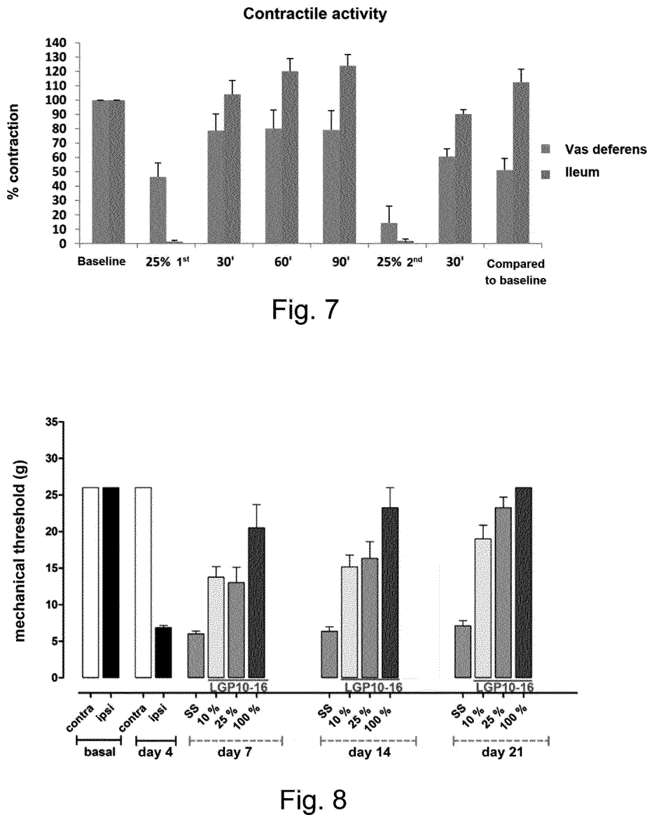

FIG. 7 shows a comparison of the results of the mouse vas deferens model and the guinea pig ileum LMMP model. In the case of the mouse vas deferens, after washing, there appears to be a sensitisation phenomenon which causes the second exposure to LGP10-16 to induce a greater response, while this process does not appear to occur in the guinea-pig ileum. The graph shows the effect of the maximum concentration of LGP10-16 (25%) in the different preparations. Before adding the drug, the baseline contraction of both preparations is, naturally, 100%. At the end of the first curve, LGP10-16 has inhibited 100% in the ileum and 55% in the vas deferens. After creating the first curve, it is washed and the ileum recovers its contractile capacity at 100%, while in the vas deferens this recovery remains at around 75%.

After the end of the second curve, LGP10-16 repeats the effect in the ileum (100% inhibition) and in this case the inhibition is greater in the vas deferens (85%). After washing the two preparations, the ileum recovers 100% of its function, while in the vas deferens, the contractile activity remains at 50% of its maximum response. The results of these in-vivo-tests suggest that LGP10-16 is capable of producing reversible changes in both the nerve conduction of peripheral sensory fibres and in functional activity, achieving complete (and reversible) inhibition of this activity at a concentration of 10% and 25%. These changes are consistent with an analgesic activity.

### Example Set 2-In Vivo

The objective of this study was to confirm the analgesic effect in an animal model for peripheric neuropathic pain. LGP10-16 was applied by subcutaneous injection, at various concentrations, in an animal model of neuropathic pain, and responses related to hyperalgesia and allodynia associated with this type of pain, as well as behavioural responses associated with animal welfare, were assessed.

The animals' response to painful stimuli in the presence of LGP10-16, their behaviour in response to maintained pain, as well as the interaction with the TLR4 receptor by means of the use of pharmacological agonists and antagonists was assessed.

Male Wistar rats with a body weight between 250 and 300 g were kept in standard laboratory conditions (12 h light/dark cycles, temperature 21° C.). Four groups were formed and treated once a day with a composition comprising an active ingredient:
Group 1 was administered 10% of LGP10-16.
Group 2 was administered 25% of LGP10-16.
Group 3 was administered 100% of LGP10-16.
A control group 4 was administered saline solution.
   Each group consisted of 15 rats.

The sciatic nerve ligation or chronic constriction injury (CCI) model for as described by Bennett and Xie was used. The sciatic nerve ligation model generates a peripheral nerve injury that causes the appearance of mechanical and thermal hyperalgesia, mechanical allodynia and sometimes signs of spontaneous pain can be observed, by the spontaneous 5 elevation of the paw. All these symptoms appear one week after nerve ligation and can last more than two months. The added value of the model is that each animal can be its own control, since the contralateral paw, not being injured, reflects normal values.

Ligation of the sciatic nerve in the rat resulted, as expected, in the development of mechanical allodynia (assessed by the von Frey filament test) and mechanical hyperalgesia (measured by the rodent pincher test). These effects were prolonged beyond 21 days.

For the realization of the control group, the identical dissection is carried out on another group of animals, with the difference that in this case the nerve is not ligated (sham group).

The nociceptive response was assessed by means of three different tests:
the pressure response test, by means of “von Frey” filaments,
the mechanical hyperalgesia, by means of the rodent pincher test,
the place preference test, as an indirect assessment of the presence of spontaneous pain.

The evaluation of the nociceptive sensitivity of the affected area was carried out using the von Frey test, which consists of the evaluation of the mechanical allodynia in the sole of the paw, by means of the application of von Frey filaments; these are rods with a nylon fiber in their tip and are calibrated to apply pressure stimuli of different magnitude (0.008-300 g). In this test, the animals were placed individually in a closed enclosure in order to apply the filaments to stimulate the area of interest. The test is carried out 5 times in each animal, leaving intervals of 3 min between each time and the different filaments are applied in increasing order of pressure, until the filament that generates a threshold pressure that induces a nociceptive behaviour in the animal is found (initiate a withdrawal movement, or vocalized). The test was carried out at different times: before surgery (basal), and at day 4 (control only), 7, 14 and 21 post-injury.

The results are summarized in FIG. 8. At day 4, the different reaction in the contralateral and the ipsilateral paw was detected. Subcutaneous administration up to the proximity of the lesion produced a dose- and time-dependent antiallodynic effect of LGP10-16, reaching statistically significant differences with respect to the control from the first run at day 7 and for all doses (10% LGP10-16, 25% LGP-10-16 and 100% LGP-10-16).

Mechanical hyperalgesia was tested in the rodent pincher test. The rodent pincher algometer allows calibrated forceps to induce quantifiable mechanical stimulation in the animal. The pressure is applied in the “inter-digital” region. The test was carried out at different times: before surgery (basal), and at 4, 7, 14 and 21 post-injury.

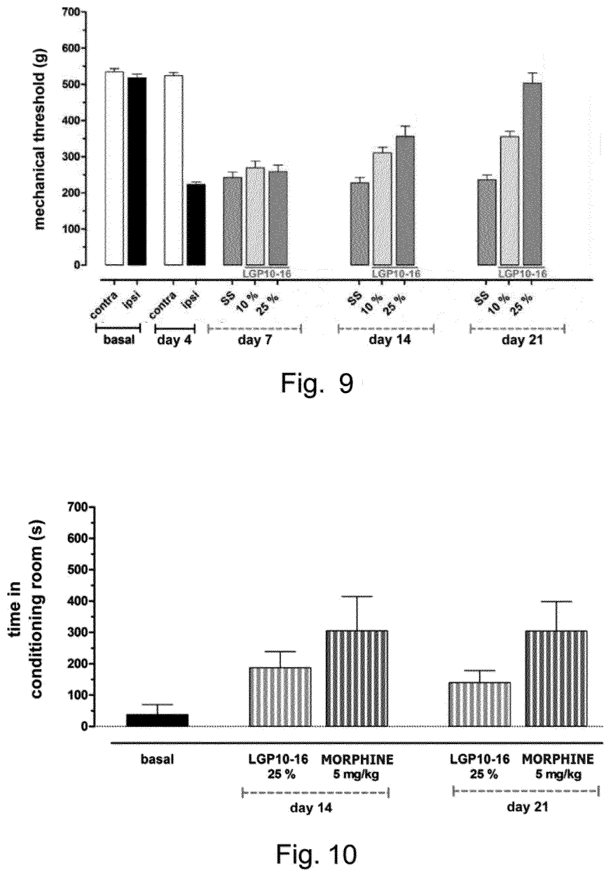

The results are summarized in FIG. 9. The antihyperalgesic effect of LGP10-16 was dose and time-dependent, reaching statistically significant differences with respect to control for all doses (10% LG P10-16, 25% LGP10-16, 100% LGP10-16) from day 14.

The conditioned place preference (CPP) test assesses emotional responses; it is based on the evaluation of an associated learning behaviour in response to a new aversive environment. The CPP apparatus (70×30×30 cm) is divided into 3 compartments: two test compartments of identical dimensions and a third one considered as a neutral zone, which is narrower. The two test compartments have different visual and tactile characteristics (colour and texture of the walls and floor), so that the animal can differentiate them well and can be isolated by movable doors, so that communication between the three compartments can be allowed or blocked. On the pre-conditioning day, the animal was allowed to roam freely in the three compartments for 30 min, and the time spent in each compartment was measured (mice that spend more than 60% in one compartment were excluded).

On conditioning days, animals were administered the drug and confined for 15 min in one of the test compartments (always in the same compartment) in order to associate the drug administration to that area. This conditioning procedure was repeated for each administration of the drug. On the day of the experiment (post-conditioning day), the animal was allowed to roam freely through the three compartments for 30 min and the time spent in each compartment was evaluated to assess whether the animal shows preference or aversion for the compartment where the administration has taken place. The CPP value is expressed as the difference between the time spent in each compartment and the time spent in each compartment. When the animal spends more time in one of them, it is associated with greater welfare, then, in our case, to less presence of spontaneous “pain”. To carry out this test, a drug with a proven analgesic effect, morphine (5 mg/kg, s.c.), was selected as a comparator.

The results are summarized in FIG. 10. Both at day 14 and day 21, the animal treated with LGP10-16 (25%) spent more time in the room associated with drug administration. This data is associated with the presence of a lower degree of discomfort (“pain”) in the room where the drug is administered.

LGP10-16 showed analgesic effect in this animal model. Healing was not negatively affected by LGP10-16 in the concentrations of 10% and 25%. However, in group 3 (100% LGP10-16) skin irritation and blistering was detected.

### Example Set 3-Formulation

The objection of this example set was the formulation of a semi-solid pharmaceutical composition for topical application comprising a volatile anaesthetic, in particular sevoflurane. A first exemplary composition “composition 1” was formulated consisting of

| | | |
|---|---|---|
| Sevoflurane | 10 | wt % |
| Isopropyl Myristate | 15 | wt % |
| Talc | 5 | wt % |
| White petroleum jelly | 66.5 | wt % |
| Cetyl alcohol | 3.5 | wt % |

The composition was manufactured comprising the following steps:
weigh the lipophilic formulation base, namely white petroleum jelly and the stabilizer, namely cetyl alcohol,
melt the formulation base and the stabilizer at 60° C., and mix to form a homogenous melt,
cool the homogenous melt to room temperature to form a basis composition,
weigh the volatile anaesthetic, namely sevoflurane, and the lipophilic solvent, isopropyl myristate, and mix,
weigh the solid carrier, namely talc, and add it to the mixture,
stir the mixture until homogenous,
add the mixture to the cool basis composition,
mix until an ointment forms,
pack in aluminium tubes.

The ointment had a pleasant texture and a homogenous structure and was stable for at least 24 hours.

A second exemplary pharmaceutical composition “composition 2” was formulated consisting of

| | | |
|---|---|---|
| Sevoflurane | 20 | wt % |
| Isopropyl Myristate | 15 | wt % |
| Talc | 5 | wt % |
| White petroleum jelly | 57 | wt % |
| Cetyl alcohol | 3 | wt % |

The second composition was manufactured according to the same method as described for the first composition. The ointment had a pleasant texture with a homogenous structure and was stable for at least 24 hours.

### Example Set 4-Comparison with Prior Art

The volatile anaesthetics can only work as analgesics if a sufficient absorption of the drug substance occurs. The absorption is only possible as long as the drug substance is in direct contact to the skin. Since the drug substances per se show a high volatility, it is the function of the formulation to keep the drug substance at the skin by reducing its evaporation. Therefore, the evaporation rate of the volatile anaesthetic from the formulation is a crucial property of the formulation.

To test this essential property of the pharmaceutical composition, the evaporation rate of a specific embodiment was measured against three prior art compositions. The following compositions were used:

The second exemplary pharmaceutical composition of example set 3 was used and labelled as S45 210101.

The ointment formulation 205 from WO 2014 143964 with sevoflurane was used (S45 210121):

| | |
|---|---|
| Propylene Glycol | 5.00% |
| Alcohol 200 Proof | 10.00% |
| 2-(2-ethoxyethoxy)ethanol | 10.00% |
| PEG-3350 | 6.00% |
| PEG-400 | 9.00% |
| Sevoflurane | 60.00% |

The translucent gel formulation 212 from WO 2014 143964 with sevoflurane was used (S45 210141):

| | |
|---|---|
| Alcohol 200 Proof | 10.50% |
| Gantrez ES-435 | 20.00% |
| Propylene Carbonate | 2.50% |
| 2-(2-ethoxytethoxy)-ethanol | 5.00% |
| Hydroxypropyl Cellulose MF | 2.00% |
| Sevoflurane | 60.00% |

The soft gel formulation 216 from WO 2014 143964 with sevoflurane was used (S45 210161):

| | |
|---|---|
| PPG-12/SMDI copolymer | 10.00% |
| Alcohol 200 Proof | 8.25% 5 |
| Gantrez ES-435 | 20.00% |
| Hydroxypropyl Cellulose GF | 1.75% |
| Sevoflurane | 60.00% |

Gantrez ES-435 is a butyl ester of PVM/MA Copolymer. It is a clear viscous solution0 containing 50% solids dissolved in propan-2-ol and butan-1-ol.

In a first experiment, tared empty blisters were used as containers for the evaporation experiment. Each blister cavity had an area of 16.5 mm*21.5 mm and a depth of 5.5 mm. The blister cavities were filled with the formulations and smoothed out. The blisters were 5 held at a temperature of 22° C. and the evaporation rate was determined by measuring the weight loss. The weight of each blister was measured at certain time intervals during the first hour every 5 minutes, during the second hour every 15 minutes, during the next two hours every 30 minutes and subsequently every hour for a total of 24 hours.

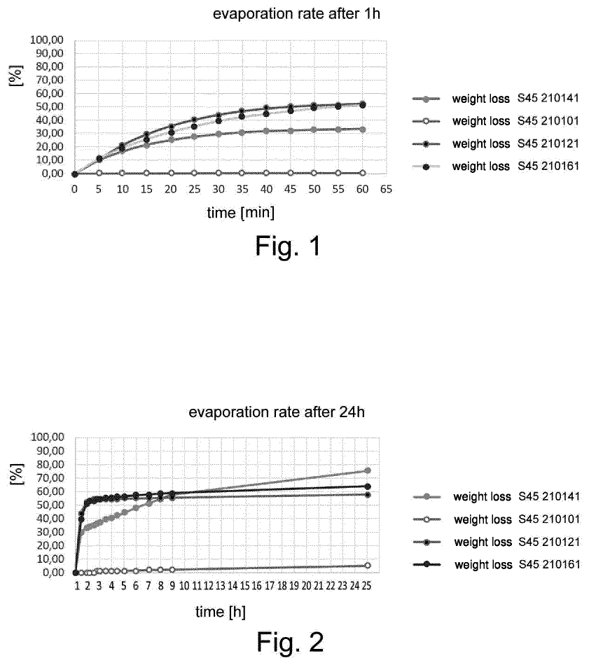

The results are summarized in FIG. 1 and FIG. 2. FIG. 1 shows that sevoflurane evaporated quickly from the prior art compositions. 30 to 50 wt % of the composition were lost within the first hour, corresponding to 50 to 80 wt % of sevoflurane. With the specific pharmaceutical composition of the present disclosure only 0.53 wt % of the composition, corresponding to 2.65 wt % of sevoflurane, evaporated in the first hour.

As can be seen in FIG. 2, after 24 hours 58 to 75 wt % of the compositions, corresponding to 75 to 100 wt % of sevoflurane, was lost from the prior art compositions, while from the specific pharmaceutical composition according to the present disclosure only 5 wt % of the composition, corresponding to 25 wt % of sevoflurane, was lost.

Especially in the prior art formulations a significant part of the Sevoflurane got already lost during the preparation of the samples. This is the reason why the total measured weight loss is lower than the total amount of volatile compounds in the formulations. Looking at the kinetic of the weight loss in FIG. 2 one can see that after about one hour almost 100% of the volatile compounds, including sevoflurane, are lost in the prior art formulations S45 210121 and S45 210161. In the formulation of the present disclosure at this point of time about 85% of the Sevoflurane are still present in the formulation.

In the prior art formulation S45 210141 the evaporation is somewhat slower, but since Sevoflurane has the highest vapour pressure of all compounds of this formulation, it is very likely that the steep evaporation course at the beginning is related to the Sevoflurane and the later flatter course to the other volatile compounds of this formulation.

Since the drug substance can only be absorbed and be effective as long as it is present at the site of action, it is crucial for the effectiveness of the dosage form to keep the drug as long at the skin as possible. Therefore, a slow evaporation rate is not only a technical property or advantage, but is highly important for the effectiveness of the drug product. In addition, a low evaporation rate reduces the dose, which is needed to achieve the therapeutic effect. By saving drug substance the costs and the environmental impact can be reduced.

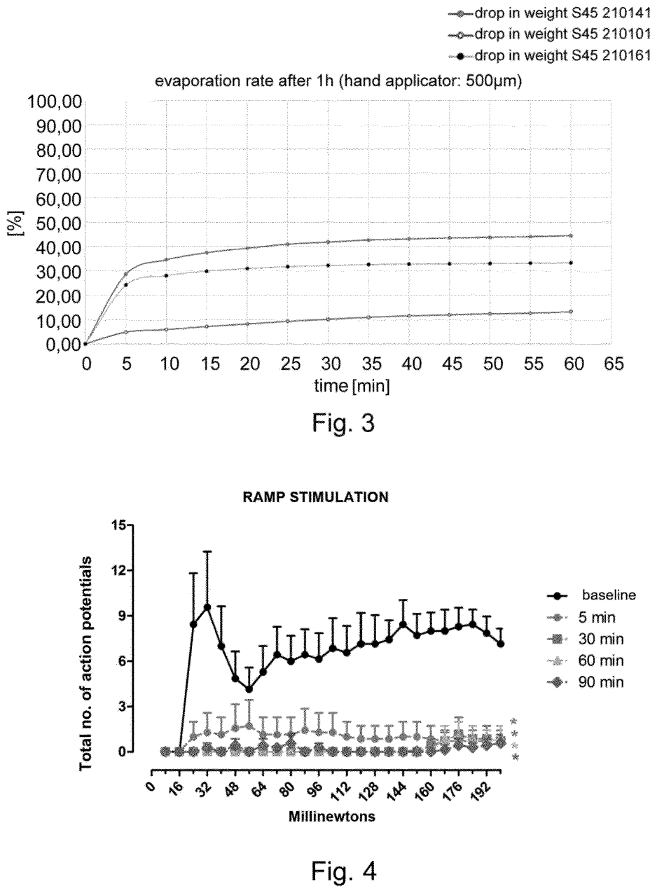

To better resemble the topical administration of the pharmaceutical composition, a second measurement was done with an ointment layer of 500 μm thickness. The results are summarized in FIG. 3. In the 500 μm layer test formulation S45 210121 is missing since the formulation was too liquid to form a stable layer. The compositions were applied to a microscope slide with a spreading knife. From this thin layer sevoflurane evaporated much faster. Since evaporation is highest at the beginning and significant evaporation already takes place while the compositions are placed on the carrier the total loss of weight that could be measured in this test was less than in the blister test. After one hour, from the prior art compositions 33 to 45 wt %, corresponding to 100 wt % of sevoflurane, had evaporated, while from the exemplary formulation only 13 wt %, corresponding to 65 wt % sevoflurane, had evaporated.

### Example Set 5-In Vivo (Topical)

The objective of this example set was to repeat example set 2 but changing the route of administration. Instead of a subcutaneous injection, the product was administered as a topical formulation (according to Example Set 3) onto the skin. The same methods were used as in example set 2.
Group 1 was administered composition 1.
Group 2 was administered composition 2.
Group 3 was administered a composition without an active ingredient (vehicle).
   Each group consisted of 15 rats.

Treatment with composition 1 showed an anti-allodynic effect that was present 30 min post administration. Treatment with composition 2 also produced a significant increase in the pressure threshold supported by the animals at 30 min post-administration, compared to the pressure supported at day 4 when the neuropathy was established. This analgesic effect was maintained until 4 h post administration. It was at 6 h post administration when the pressure supported by the animals was similar to that of the vehicle animals and of that of day 4. On the second day of treatment similar results are revealed.

Continuous administration (twice a day from day 5 to day 21) provided a reversion of allodynia suffered by the animals, since pressure values returned to baseline level at day 14, which was maintained until day 21. This effect was maintained for 96 h after the last administration. No side effects were detected, nor modification of locomotor activity. No side effects or adverse reactions have been reported for composition 1 and composition 2 during the whole study.

The results demonstrated that the present disclosure provides an effective, well-tolerated treatment option for pain, in particular a treatment option that is cost efficient and environmentally sustainable.

### Example Set 6-In Vivo (Topical)

The objective of this example set was to analyse, if LGP10-16 has the same effect on female rats.

18 adult female and 18 adult male Wistar rats (240-300 g) were used.

CCI (chronic constriction injury) model was used (Bennett & Xie, 1998). The animals were allowed to recover from surgery for 24 hrs. The contralateral leg was used as control, and a sham group was also included.

Composition 2 was topically applied, using a template to standardize the total amount of drug administered. The final area of administration was around 6 cm². LGP10-16 effects were evaluated by administering composition 2 according to example set 3 or placebo formulation. The experiments began one week after surgery and drug administration was carried out once a day, starting on day 6 of the protocol on a daily basis up to day 14.

Tactile allodynia was assessed, through the von Frey test as previously described (Pascual et al., 2005). This test was carried out prior to CCI surgery to obtain basal values, and on day 4 post surgery. The test was repeated on day 14 at 30, 60, 90, and 240 min post administration.

Treatment with composition 2 produced a significant increase in the pressure threshold tolerated by the animals at 30 min post-administration, compared to the pressure tolerated at day 4.

Treatment with composition 2 induced a conditioning effect towards the chamber paired with drug administration, with a significant increase in the time spent in the room paired to the drug. This indicates a preference of the animal to stay in the place where drug was administered, suggesting a comfortable effect of the drug. This conditioning occurred for both sexes, male and female. Hence, there is no sex difference in the emotional pain relief due to composition 2.

1. A pharmaceutical composition for topical administration, comprising:
at least one volatile anaesthetic;
at least one pharmaceutically acceptable lipophilic solvent; and
a lipophilic formulation base.

2. The pharmaceutical composition according to claim 1, wherein the volatile anaesthetic is selected from the group consisting of isoflurane, halothane, enflurane, sevoflurane, desflurane, methoxyflurane, and mixtures thereof.

3. The pharmaceutical composition according to claim 1, wherein the pharmaceutical composition comprises up to 40% by weight of the volatile anaesthetic.

4. The pharmaceutical composition according to claim 1, wherein the volatile anaesthetic comprises sevoflurane.

5. The pharmaceutical composition according to claim 1, wherein the lipophilic formulation base is a semi-solid ointment base.

6. The pharmaceutical composition according to claim 1, wherein the pharmaceutical composition is a single-phase formulation.

7. The pharmaceutical composition according to claim 1, wherein the lipophilic solvent comprises an ester of a fatty acid selected from the group consisting of myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, caprylic acid, capric acid, lauric acid, myristic acid, palmitic acid, stearic acid, arachidic acid, behenic acid, lignoceric acid, cerotic acid, and any combinations thereof,
and/or wherein the lipophilic solvent is an ester of a C₁ to C₆ alcohol.

8. The pharmaceutical composition according to claim 1, wherein the pharmaceutical composition comprises up to 45% by weight of the lipophilic solvent.

9. The pharmaceutical composition according to claim 8, wherein the lipophilic solvent comprises isopropyl myristate.

10. The pharmaceutical composition according to claim 1, wherein the lipophilic formulation base comprises a pharmaceutically acceptable semi-solid hydrocarbon compound or mixture of hydrocarbon compounds with a drop point between 35° C. und 70° C.

11. The pharmaceutical composition according to claim 1, wherein the pharmaceutical composition comprises from 35% by weight to 90% by weight of a pharmaceutically acceptable semi-solid hydrocarbon compound or mixtures of hydrocarbon compounds.

12. The pharmaceutical composition according to claim 1, wherein the lipophilic solvent works as a solubilizer, enabling the solution of the volatile anaesthetic in the lipophilic formulation base.

13. The pharmaceutical composition according to claim 1, wherein the pharmaceutical composition comprises at least one pharmaceutically acceptable stabilizer in a concentration of from 0.1% by weight to 5% by weight.

14. (canceled)

15. The pharmaceutical composition according to claim 1, wherein the pharmaceutical composition comprises a C₁₀ to C₃₀ fatty alcohol.

16. The pharmaceutical composition according to claim 1, wherein the pharmaceutical composition comprises at least one carrier selected from the group consisting of kaolin, bentonite, hectorite, colloidal magnesium, aluminum silicate, silicon dioxide, magnesium trisilicate, aluminum hydroxide, magnesium hydroxide, magnesium oxide, and talc.

17. The pharmaceutical composition according to claim 16, wherein the pharmaceutical composition comprises from 1% by weight to 15% by weight of the carrier.

18. A pharmaceutical composition comprising:
Sevoflurane, in a concentration of from 10% by weight to 25% by weight;
Isopropyl Myristate, in a concentration of from 10% by weight to 20% by weight;
Talc, in a concentration of from 3% by weight to 7% by weight;
Petroleum jelly, in a concentration of from 55% by weight to 75% by weight; and
Cetyl alcohol, in a concentration of from 2% by weight to 5% by weight.

19. (canceled)

20. (canceled)

21. (canceled)

22. (canceled)

23. (canceled)

24. (canceled)

25. (canceled)

26. (canceled)

27. (canceled)

28. A method for the treatment of pain, wherein sevoflurane is topically administered in form of a pharmaceutical composition according to claim 1.

29. The method according to claim 28, wherein after administration of the pharmaceutical composition, the site of administration is covered by an occlusion-producing layer.

30. (canceled)

31. (canceled)

32. (canceled)

33. (canceled)

34. (canceled)

35. (canceled)

36. The method according to claim 28, wherein the sevoflurane is applied every 12 to 24 hours.
